(19) United States
(12) Patent Application Publication
(10) Pub. No.: US 2007/0178142 A1
(43) Pub. Date: Aug. 2, 2007

(54) Animal food package with chewable scoop to promote dental health

(76) Inventor: Brett C. Titus, Parker, CO (US)

Correspondence Address: HOLLAND & HART, LLP, P.O BOX 8749, DENVER, CO, 80201, US

(21) Appl. No.: 11/341,971

(22) Filed: Jan. 27, 2006

(51) Int. Cl. A61K 8/96; A61K 8/66

(52) U.S. Cl. 424/442; 424/50

(57) ABSTRACT

An edible animal food scoop formulated to promote dental health is provided. The edible animal food scoop is used to dispense animal food from a container and, typically when the container is empty, is provided to the animal for chewing or consumption. Chewing the edible animal food scoop facilitates dental health.

### FIELD OF THE INVENTION

The present invention relates to animal food and, more particularly, to containers of dog food with chewable scoops to promote dental health.

### BACKGROUND OF THE INVENTION

In recent years, veterinarians and pet owners have become more educated and responsible regarding dental care for their pets. Today, for example, many pets receive dental care and cleaning from veterinarians. Also, veterinarians provide and promote dental care through many products such as brushing, rinsing and the like.

Some pet owners and pets, however, do not like brushing, rinsing and the like. To accommodate these dogs, enterprising companies have developed a variety of chews and toys to promote and enhance the dental well being of pets. For example, some toys are designed with fibers to simulate in a dog the action of floss while the dog chews the toy, see for example, U.S. Pat. No. 5,215,038, titled Dog Chew Toy, issued to O'Rourke, and incorporated herein by reference. Other chews also are available. For example, U.S. Pat. No. 6,050,224, titled Therapeutic Chew Device for Cleaning Teeth and Breath of Dogs, issued to Owens, relates to a chew with activated charcoal, which is incorporated herein by reference.

Many other chews exist on the market today. Many of which provide some dental cleaning action. Even conventional rawhides provide some cleaning and other dental healthcare. One popular style of treat is marketed as GREENIES®, from S & M NuTec, LLC.

While some of these products function to clean animals teeth, they are often given infrequently as owners either remember or have access to the products. Thus, it would be desirous to provide an improved teeth cleaning device.

### SUMMARY OF THE INVENTION

To attain the advantages and in accordance with the purpose of the invention, as embodied and broadly described herein, an animal dental care package is provided. The animal dental care package includes a container of animal food with an edible dispensing cup provided. The edible dispensing cup is used to dispense food and is given to the animal to eat once the container of food is empty. The edible dispensing cup is formulated to promote dental health of the animal.

The present invention also provides methods for dental care of an animal. The method includes providing animal food in a container with an edible food scoop specifically configured to promote dental well being in the animal. Using then edible food scoop to dispense food. After dispensing food, providing the edible food scoop to the animal for consuming by the animal to promote dental health.

The foregoing and other features, utilities and advantages of the invention will be apparent from the following more particular description of an embodiment of the invention as illustrated in the accompanying drawings. Further, the advantages and purpose of the invention will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments of the present invention, and together with the description, serve to explain the principles thereof. Like items in the drawings are referred to using the same numerical reference.

FIG. 1 shows a perspective view of an edible animal food scoop in a container of animal food; and

FIG. 2 shows a flowchart illustrating a method of using an edible dog scoop.

### DETAILED DESCRIPTION

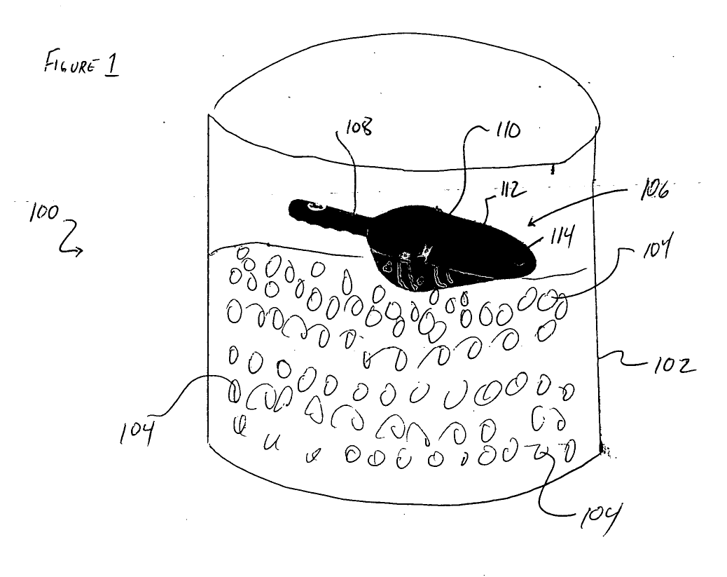

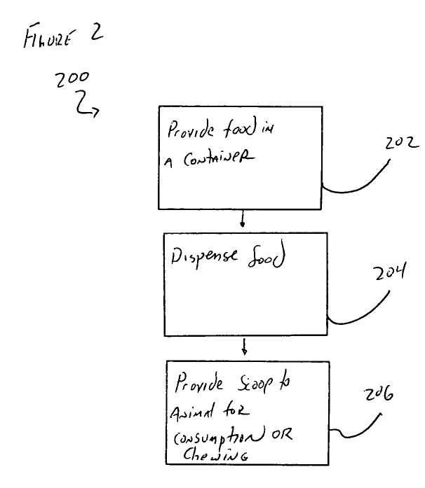

The present invention will now be explained with reference to FIGS. 1 and 2. While the present invention is particularly described with relation to dog food, one of ordinary skill in the art will understand on reading the disclosure that the present invention could apply to any animal food and animal dental care.

Referring to FIG. 1, a dental care system 100 is shown. Dental care system 100 includes a food container 102 filled with animal food 104, such as, for example, kibble for dogs. Container 102 may be a stand alone container to which food 104 is separately added from time to time or a bag of food, such as a standard bag of food available from any number of manufacturers including, for example, Hills Pet Nutrition, Inc., Ralston Purina Company, the Iams Company, or the like.

System 100 includes an edible dog food scoop 106. Edible dog food scoop 106 includes a grip 108 and a cup 110. Grip 108 may be a handle as shown. Further, grip 108 may be decoratively formed or shaped to facilitate holding the scoop. Grips other grips than a handle are possible. Cup 110 may be sized to provide a particular amount of food 104 in each scoop, such as ½ cup, 1 cup, 2 cups or the like. Alternatively, cup 110 may have indicia 112 providing graduation or the like to measure out food amounts.

While dog food scoop 106 may be provided individually, it is envisioned that a bag of dog food will be provided with one or more edible scoops 106. Scoop(s) 106 and dog food would be maintained in the bag of dog food, where the bag is container 102, or transferred to a separate container 102.

Edible dog food scoop 106 may be formulated similar to any conventional dental cleaning products, such as, for example, rawhide and more particularly rawhides coated with enzymes, abrasives and the like such as rawhides, C.E.T.® rawhides from Virbac Corporation, GREENIES®, biscuits such as Merrick's dental treats from Merrick Pet Care, Ltd., other composite dental product and the like. The edible scoop also may contain additives 114 to help promote teeth and gum health, such as, for example, fluoride, activated charcoal, fibers, abrasives, or the like.

Referring now to FIG. 2, a flowchart 200 is provided illustrating a method of dental care for a dog. First, dog food is provided in a container, step 202. Next, dog food is dispensed using an edible dog food scoop configured to promote dental health in the dog, step 204. As one of ordinary skill in the art will now recognize on reading the disclosure, using the edible dog food scoop to dispense the food accomplishes multiple tasks. One of the tasks relates to providing the edible dog food scoop with some flavor of the dog food as it comes in regular contact with the dog foods. This makes it more likely that the dog will eat the scoop when it is presented to the dog. To increase the flavor absorption, optionally, the dog food scoop may be stored in the container with the food. Alternatively, the scoop may be stored external to the container. Also, using the scoop avoids the use of make shift alternative dispensing apparatuses, such as, for example, fast food containers, tin cans, soda or pop cans, and the like that many dog owners use to dispense dog food. Many of these containers contain food substances that may not be healthy for the dog, and may provide additional calories causing obesity and accelerate any dental deterioration. After dispensing dog food, the edible dog food scoop is provided to the dog for consumption (or at least chewing if the animal is not allowed to eat the scoop) to facilitate teeth cleaning and other dental care, step 206. It is envisioned the scoop will be provided as a treat when the container of food is empty. The dog owner will be provided a new edible scoop when a new container of food is purchased. In some instances, such as, for example, large bags of food, several scoops may be provided in a single container so the scoops can be given more frequently than once a container. The number of scoops could be provided so the scoops can be given out at predetermined intervals, such as, for example, a bag of food may container 3 scoops so a scoop can be provided as a treat to clean teeth every ⅓ when ⅓ of the bag of food is finished. Although providing the scoop can be done whenever desired.

While the invention has been particularly shown and described with reference to some embodiments thereof, it will be understood by those skilled in the art that various other changes in the form and details may be made without departing from the spirit and scope of the invention.

1. An animal dental care system, the system comprising:
a container;
animal food contained in the container; and
an edible animal food scoop formulated to promote dental health, wherein providing the edible animal food scoop to the animal for consumption promotes the dental health of the animal.

2. The system of claim 1, wherein the container is a bag of animal food.

3. The system of claim 2, wherein the edible animal food scoop is packaged with the bag of animal food.

4. The system of claim 3, wherein the edible animal food scoop is packaged internal to the bag of animal food and in contact with the animal food.

5. The system of claim 1, wherein the animal food is dog food.

6. The system of claim 5, wherein the edible animal food scoop is an edible dog food scoop comprising a dog chew, and the dog chew is selected from a group of chews consisting of: rawhides, biscuits, and composites.

7. The system of claim 1, wherein the edible animal food scoop comprises additive to promote dental health.

8. The system of claim 7, wherein the additives are selected from the group of additives consisting of: abrasives, fluorides, and enzymes.

9. The system of claim 1, wherein the edible animal food scoop comprises a cup and a handle.

10. The system of claim 1, wherein the edible animal food scoop comprises a cup of a selected size to measure out a quantity of animal food.

11. The system of claim 10, wherein the size is selected from a group of sizes consisting of: ¼ cup, ⅓ cup, ½ cup, ⅔ cup, ¾ cup, 1 cup, 1.25 cups, 1.3 cups, 1.5 cups, 1.6 cups, 1.75 cups, 2 cups, 2.25 cups, 2.3 cups, 2.5 cups, 2.6 cups, 3 cups, 3.25 cups, 3.3 cups, 3.5 cups, 3.6 cups, 3.75 cups, and 4 cups.

12. The system of claim 1, wherein the edible animal food scoop comprises indicia to facilitate dispensing a determinable amount of animal food.

13. The system of claim 12, wherein the indicia designate amounts of food including ¼ cup, ⅓ cup, ½ cup, ⅔ cup, ¾ cup, 1 cup, 1.25 cups, 1.3 cups, 1.5 cups, 1.6 cups, 1.75 cups, 2 cups, 2.25 cups, 2.3 cups, 2.5 cups, 2.6 cups, 3 cups, 3.25 cups, 3.3 cups, 3.5 cups, 3.6 cups, 3.75 cups, and 4 cups.

14. A method of dental care for animals comprising the steps of:
providing a container of animal food;
providing an edible food scoop to dispense food from the container to feed the animal;
dispensing animal food to the animal; and
feeding the animal the edible food scoop, wherein eating the edible food scoop promotes dental health of the animal.

15. The method of claim 14, wherein the container is a bag of dog food and the edible food scoop is provided in the bag of dog food.

16. The method of claim 14, further comprising the step of:
storing the edible food scoop in the container of animal food; and
absorbing a flavor of the animal food into the edible food scoop.

17. An edible animal food scoop to promote dental health in the animal, the edible animal food scoop comprising:
a grip to facilitate using the food scoop to dispense animal food from a container of animal food;
a cup attached to the grip, the cup to dispense an amount of animal food from the container of animal food; and
the edible animal food scoop is formulated to promote dental health, wherein providing the edible animal food scoop to the animal for consumption promotes the dental health of the animal.

18. The edible animal food scoop of claim 17, wherein the edible animal food scoop is an edible dog food scoop comprising a dog chew, and the dog chew is selected from a group of chews consisting of: rawhides, biscuits, and composites.

19. The edible animal food scoop of claim 17, wherein the edible animal food scoop comprises additive to promote dental health.

20. The edible animal food scoop of claim 19, wherein the additives are selected from the group of additives consisting of: abrasives, fluorides, and enzymes.
